(19) United States
(12) Patent Application Publication
(10) Pub. No.: US 2008/0294255 A1
(43) Pub. Date: Nov. 27, 2008

(54) Sinus Tube

(76) Inventor: Donald Albert Gonzales, San Antonio, TX (US)

Correspondence Address: FULBRIGHT & JAWORSKI L.L.P., 600 CONGRESS AVE., SUITE 2400, AUSTIN, TX, 78701, US

(21) Appl. No.: 11/744,566

(22) Filed: May 4, 2007

Related U.S. Application Data
(60) Provisional application No. 60/802,758, filed on May 23, 2006.

(51) Int. Cl. A61F 2/18; A61M 39/02

(52) U.S. Cl. 623/10; 606/108

(57) ABSTRACT

The inventive sinus tube provide a non-surgical approach to sinus disease. The tube once inserted in the ostia of a paranasal sinus provides the proper aeration and drainage of the sinus cavity. The tube stops the cycle of inflammation, stasis, infection, and continued inflammation associated with sinus disease. The tubes are inserted into the natural ostia of a patient or are inserted after a surgical procedure to enlarge the ostia. The invention also provides an instrument for inserting the inventive tube, methods of using the tube, and kits including the tubes.

### RELATED APPLICATIONS

The present application claims priority under 35 U.S.C. § 119(e) to U.S. provisional patent application, U.S. Ser. No. 60/802,758, filed May 23, 2006; which is incorporated herein by reference.

### BACKGROUND OF THE INVENTION

Sinusitis is a progression of inflammation, stasis, infection, and continued inflammation. Typically, the beginning of all sinus infections is either allergy or viral infection. Both of these conditions lead to swelling of the sinus and nasal mucosa, that when severe enough, causes the small holes, called ostia, of the sinuses to close. Once the ostia is closed, the environment inside the sinuses, specifically the maxillary, sphenoid, and frontal sinuses, becomes conducive to microbial growth. The way this typically occurs is that once the ostia is shut the oxygen content of the sinus drops, and the fluid inside the sinus is unable to escape which leads to further inflammation. Furthermore, the reduced oxygen content and inflammation disrupts the ability of the cilia of the sinus cells to operate properly which leads to further stasis.

The typical patient that is seen by the otolaryngologist is started on antibiotics. Usually the antibiotic course can be as long as six weeks to eradicate the bacteria and bring the sinuses back to normal. For those patients in which antibiotics do no relieve the problem, the only alternative is surgery. Although sinus and nasal surgeries are now common with 500,000 to 700,000 of such surgeries being performed annually in the U.S., these surgeries are typically both destructive and permanent. The openings of the sinuses are typically enlarged with a biting instrument which causes irreversible changes to the sinuses and the ostia leading to the sinuses. Around 10% of patients who undergo sinus surgery have scarring that leads to continued sinus problems which frequently require revision surgery.

Given the frequent complications and sequelae of sinus surgery, there remains a need in the art for keeping the ostia of the sinuses open without the risks and complications associated with sinus surgery. The desired solution preferably limits or eliminates the need for sinus surgery which can result in infection, scar tissue formation, adhesions, bleeding, later sinus problems, and patient discomfort.

### SUMMARY OF THE INVENTION

The present invention provides a system for keeping open the ostia leading to the paranasal sinuses in a subject. The system provides for pressure equalization of the sinuses as well as proper aeration and drainage of these spaces. In certain embodiments, the placement of an inventive tube is an alternative to surgery. In other embodiments, it is complementary to surgery. The tubes act similarly to the tubes used commonly for pressure equalization placed in the tympanic membranes of the ears of children. The tubes keep the ostia leading to the paranasal sinuses, particularly the maxillary sinuses, open for aeration and proper drainage of the sinus. The placement of tubes aids the eradication of a microbial infection and prevents the occurrence of future sinus problems in the patient. The sinus tube may also be a depot for the release of a pharmaceutical agent such as an antibiotic or anti-inflammatory agent in the sinus and/or nasal cavity.

In one aspect, the invention provides a sinus tube for placement in the ostia of a paranasal sinus. The tube is typically made of a biocompatible material and may optionally be bioabsorbable. A picture of an exemplary sinus tube is shown in FIGS. 1 and 5-27. The tube is a tubular structure with a lumen and an opening on each end. The tube is approximately 2 mm in length. As would be appreciated by one of skill in this art, the tubes may come in different sizes for different patients ranging from infants to adults. Surrounding each opening is preferably a flange or ridge for stabilizing the tube once it has been inserted into the ostia. The ridge or flange around the opening being inserted into the sinus is typically smaller the ridge or flange on the opening on the opposite side. This allows for easy insertion of the tube into the ostia of the patient. For example, the inner opening may have a small ridge or bump (e.g., less than approximately 0.3 mm in height) around it to prevent the tube from falling out too easily after insertion. The outer flange may be larger in order to prevent the tube from falling into the sinus. In some embodiments, the tube is symmetrical, and the flanges or ridges on each side are the same. Various designs of the sinus tube are described herein, and other designs are possible without going beyond the scope of the present invention.

The inventive tubes are also optionally coated. The coating may include a timed release formulation of a pharmaceutical agent such as an anti-inflammatory agent, a steroid, decongestant, antibiotic, etc. In certain embodiments, the tube may be coated to prevent adhesion of the tube in place. In other embodiments, the tube may be coated with a material suitable for cell growth. In yet other embodiments, the tube may be coated with a material to prevent cell growth such as a cytotoxic agent. The tube may also be coated to make the device more biocompatible. Many coatings for medical devices are known in the art.

The invention also provides a method for inserting the inventive sinus tubes into the ostia of a paranasal sinus. The tube may be placed in the natural ostia of the sinus or the tube may be placed after surgery or a procedure to enlarge the ostia. The tube can be placed in the subject at a physician's office or during surgery in an operating room. Typically the ostia is visualized and the tube is placed using an instrument for gripping and then releasing the tube once it is in place. See FIG. 4. In certain embodiments, the whole tube or the inner flange of the tube is deformable to allow for easy placement of the tube. The tube is preferably designed to regain its original shape after it has been released or deformed. The tube is left in place to provide proper aeration and drainage of the paranasal sinus for a sufficient time to be determined by the physician treating the patient. Later, after there is no longer a need for the tube, it may be removed manually. However, preferably the tube falls out of place into the nasal cavity and is harmlessly swallowed by the patient. The tube is then degraded and/or eliminated by the digestive tract of the patient.

One of the advantages of the sinus tube is that it offers physicians an alternative to long course antibiotics and traditional sinus surgery. The tube allows for proper aeration of the sinus which provides an environment that is not conducive to bacterial growth. The tube also provides a nondestructive alternative to surgery by not requiring any permanent change to the sinuses, ostia, or nasal passage, which may lead to later complications for the patient. For example, the use of a tube in lieu of surgery significantly reduces the risk of scarring which can lead to lifelong sinus problems.

In another aspect, the invention provides an instrument for inserting the inventive sinus tube into the ostia of a paranasal sinus. The instrument typically includes a comfortable grip and an elongated end with a means for holding and releasing the inventive sinus tube in place. The instrument may radially compress or deform the inner flange of the tube in order to allow for easy insertion of the device into the ostia of a patient. Once the tube is in place, it is released from the instrument. The invention also provides an instrument for removing the tube.

In another aspect, the invention provides a kit including the inventive sinus tube. The kit may also include multiple sizes of the sinus tube, pharmaceutical agents (e.g., steroids, antibiotics), an instrument for inserting the tube, an instrument for removing the tube, instructions for inserting the tube, etc. Typically, these items are conveniently packaged for the use by a treating physician. In certain embodiments, the items are sterilely packaged.

The present invention provides a new system for treating sinus disease. The sinus tubes provide proper aeration and drainage of the sinus cavities without the need for destructive surgical procedures. Thus, the inventive system eliminates the risks associated with surgery such as scar formation, adhesions, bleeding, infection, and future sinus problems.

### BRIEF DESCRIPTION OF THE DRAWING

FIG. 1 shows a three quarter view and a side view of a sinus tube.

FIG. 2 shows an axial view of the nasal cavity with a sinus tube in place and a view from inside the nasal cavity.

FIG. 3 is a frontal view of variously shaped flanges of the inventive sinus tubes.

FIG. 4 is a drawing of an instrument used to insert the inventive sinus tubes. The drawing also includes a close up of the tip with the mechanism for holding the tube and releasing it in place. A tube on the instrument is also shown.

FIG. 5 is a drawing of an exemplary sinus tube made from a mesh material.

FIG. 6 is a drawing of an exemplary sinus tube with no flanges.

FIG. 7 is a drawing of an exemplary sinus tube with flanges and oval openings.

FIG. 8 is a drawing of an exemplary sinus tube with a square cross-section.

FIG. 9 is a drawing of an exemplary sinus tube with a square cross-section but a round lumen.

FIG. 10 is a drawing of an exemplary sinus tube with circular ridges.

FIG. 11 is a drawing of an exemplary sinus tube with asymmetrical flanges.

FIG. 12 is a drawing of an exemplary sinus tube with asymmetrical flanges and holes.

FIG. 13 is a drawing of an exemplary sinus tube with symmetrical flanges.

FIG. 14 is a drawing of an exemplary sinus tube with symmetrical flanges and holes.

FIG. 15 is a drawing of an exemplary sinus tube with symmetrical broken flanges.

FIG. 16 is a drawing of an exemplary sinus tube with asymmetrical broken flanges.

FIG. 17 is a drawing of an exemplary sinus tube with asymmetrical broken flanges and holes.

FIG. 18 is a drawing of an exemplary sinus tube with symmetrical broken flanges and holes.

FIG. 19 is a drawing of an exemplary sinus tube with a tapered (sloped) insertion flange.

FIG. 20 is a drawing of a threaded version of a sinus tube.

FIG. 21 is a drawing of an exemplary sinus tube with symmetrical flanges and slots.

FIG. 22 is a drawing of an exemplary sinus tube with asymmetrical protrusions.

FIG. 23 is a drawing of an exemplary sinus tube with symmetrical protrusions.

FIG. 24 is a drawing of an exemplary sinus tube with an hourglass shape.

FIG. 25 is a drawing of an exemplary sinus tube with mesh walls and symmetrical flanges.

FIG. 26 is a drawing of an exemplary sinus tube with flanges.

FIG. 27 is a drawing of an exemplary sinus tube.

### DETAILED DESCRIPTION OF THE INVENTION

The present invention provides a system for keeping open ostia of a paranasal sinus utilizing a tubular structure in order to provide for proper aeration and drainage. In the case of many diseases affecting the sinuses from infection to allergies to surgery, the tissue around the ostia becomes inflamed and swollen leading to the closing off of the sinus cavity to the nasal passage. The resulting lack of drainage and aeration provides an environment conducive to infection. Infection then leads to further inflammation. Thus begins the cycle of sinus disease. In order to prevent this undesired outcome, the present invention provides sinus tubes, similar to the pressure equalization tubes placed in the tympanic membranes of the ears of children. These tubes are preferably used in lieu of any surgery; however, the tubes may also be used in conjunction with surgery. The sinus tubes may also be used to deliver a pharmaceutical agent to the sinus or nasal passage by impregnating the tube itself or a coating on the tube with the agent to be delivered. The invention not only provides the sinus tubes but also provides kits, instruments for placing and removing the tubes, and procedures for inserting and removing the inventive tubes.

A patient suffering from sinus or nasopharynx disease (e.g., allergies, inflammation, infection) or a patient who has had sinus or nasal surgery is a candidate for the placement of the inventive sinus tubes. The placement of a tube in the ostia of a sinus prevents the closing up of the ostia. Keeping the ostia open allows for the proper aeration and drainage of the sinus cavity. This leads to a healthier environment in the sinus cavity and promotes the proper resolution of any inflammation or infection. The sinus tube may be placed in a patient at any time. The tube may be inserted in a doctor's office or in an operating room. Preferably, the tube is inserted into the ostia before the ostia becomes inflamed and swollen. The tube may also be placed at the time a sinus infection is diagnosed. After the tubes are no longer needed, the tube may fall out of place and be harmlessly swallowed by the patient, or the tube may be manually removed. In certain embodiments, once the sinus disease has subsided, the tube automatically falls out of place and is harmlessly swallowed by the patient. Alternatively, the tube is made of biodegradable, bioabsorbable materials that is absorbed by the patient's body over time.

The sinus tube may also be placed in the Eustachian tube of a patient. In certain embodiments, the patient is a pediatric patient. The sinus tube can be placed in the Eustachian tube from the nasopharynx. Placement of the tube allows for pressure equalization between the middle ear and the nasopharynx. The tube may be inserted for the treatment of inflammation, allergies, infection, or other Eustachian tube dysfunction. In certain embodiments, the sinus tube is inserted to treat otitis media.

The sinus tubes may stay in place from 1 week to 6 months depending on the judgment of the treating physician. In certain embodiments, the tubes are kept in from 2 weeks to 8 weeks, or 3 weeks to 6 weeks. In other embodiments, the tubes are kept in place for approximately 1 month, 2 months, 3 months, 4 months, 5 months, or 6 months. In certain embodiments, the tube may be kept in longer or even permanently. The tube may be visualized periodically to confirm that the tube is still in place and functioning properly. If a tube has been found to have fallen out, a new tube may be inserted in its place. Of course, as would be appreciated by one of skill in the art, if the tube is no longer needed a new tube would not be inserted. In certain embodiments, the material the tube is made of may determine how long the tube is kept in. For example, the tube may be designed to degrade over 2 weeks to 6 months. The tube may be needed by the patient for less time than it takes the tube to degrade. For example, the tube may only be needed to 3-4 weeks but take 3-6 months to degrade.

The sinus tube is a tubular structure designed to be inserted into the ostia of a paranasal sinus in order to keep the ostia open. The tube ranges in length from 0.5 mm to 5 mm, preferably approximately 2 mm in length. Tubes for children may be smaller. The diameter of the tubular structure ranges from 0.5 mm to 5 mm, preferably approximately 3 mm. The tube has a lumen and an opening at each end. One end fits into the sinus, and the other end remains out in the nasal passage. The openings and lumen provide for the free follow of air and secretions from the sinus into the nasal. The cross-sectional shape of the tube may be any shape that provides for a lumen which allows air and secretions to flow through it. Typically, the tube is cylindrical with a circular cross-sectional shape. However, the cross-section may be polygonal (e.g., triangular, rectangular, square, pentagonal, hexagonal, octagonal, etc.), oval, etc. The tube may also be uniform throughout its length, or it may change shape and/or size. For example, the end of the tube in the sinus cavity may be smaller than the end in the nasal cavity.

The tube is made of a material sufficient to resist it being closed off by inflammation or swelling of the tissue around the ostia. That is, the tube is preferably able to withstand the circumferential force applied to it by the surrounding tissue without substantial collapse of the lumen of the tube. The tube may include ribbing, arches, rings, spirals, or other structures to provide strength to the tube. The tube may be made of one material or multiple materials.

The tube may have solid walls, or the walls of the tube may include openings. The openings may be as simple as holes in the wall of the tube. Or the wall of the tube may include more complex shapes such as lines, spirals, slots, irregular shapes, etc. In certain embodiments, the walls of the tube are made of a mesh material. Openings in the wall of the tube may provide for better drainage of the sinus cavity.

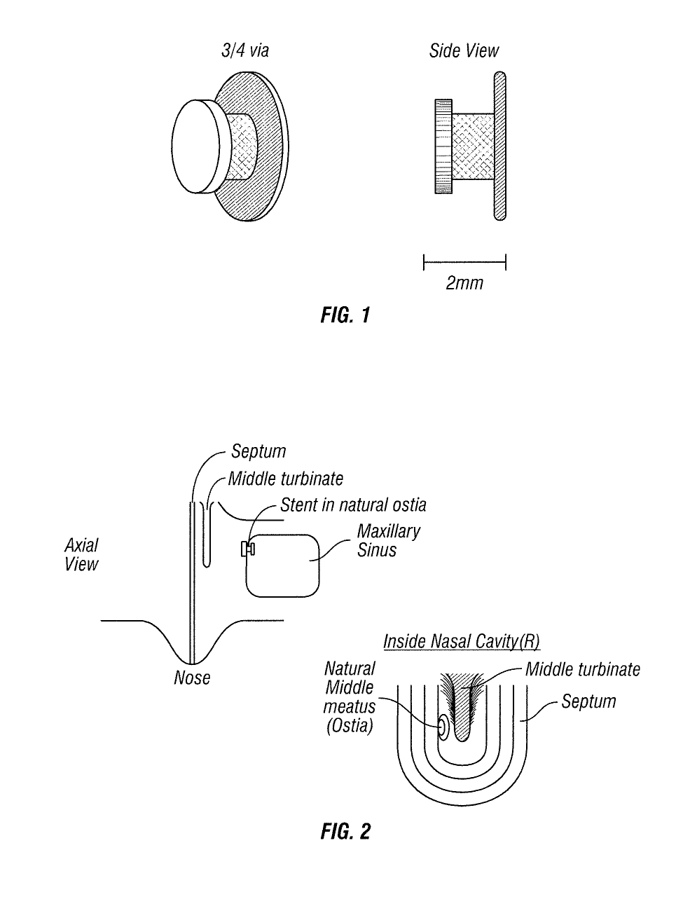

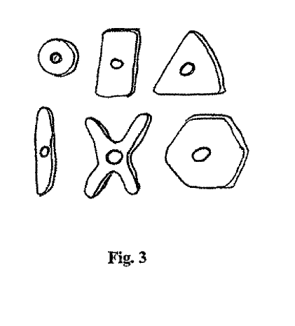

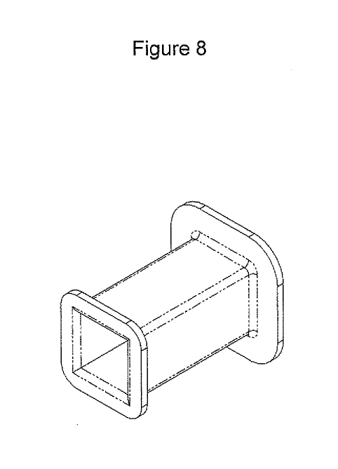

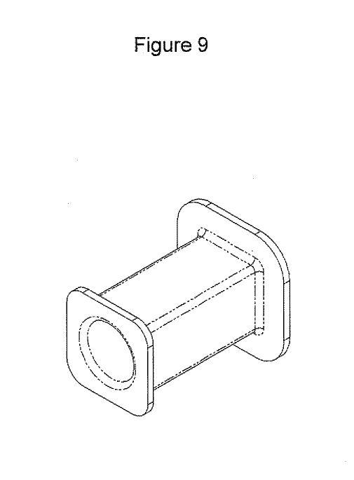

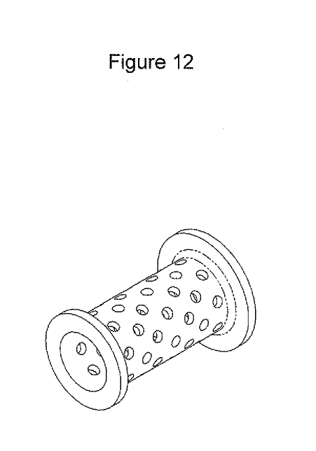

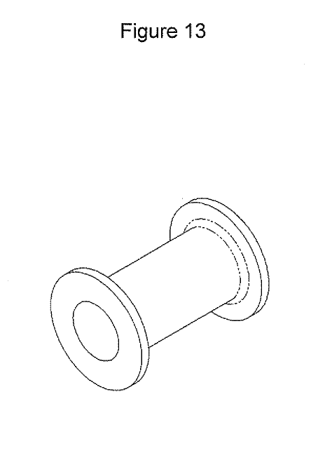

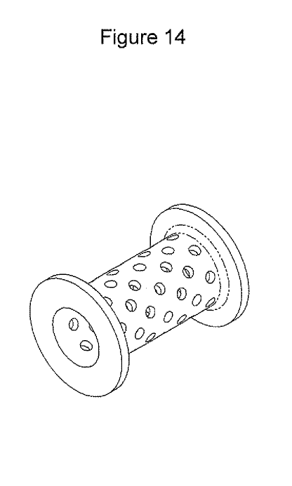

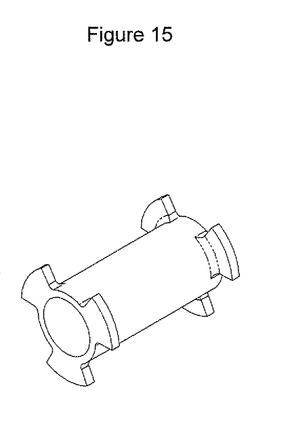

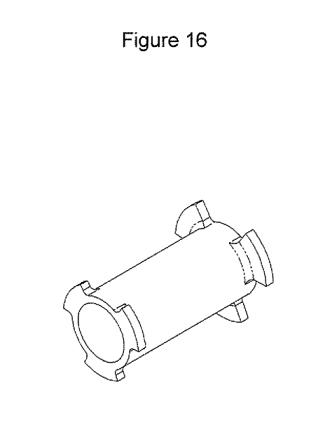

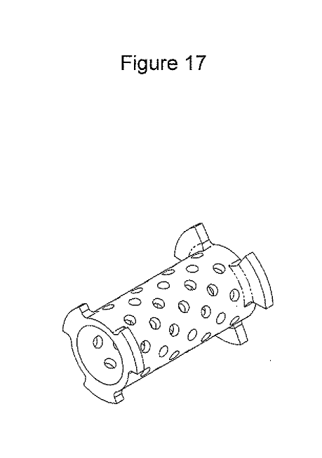

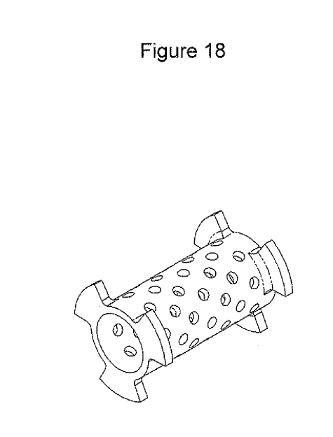

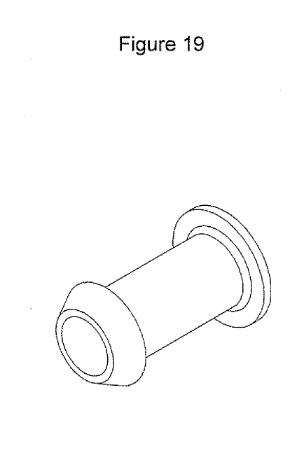

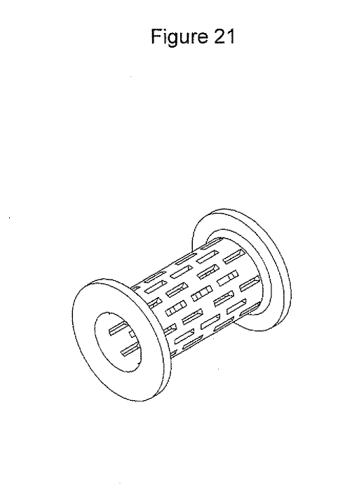

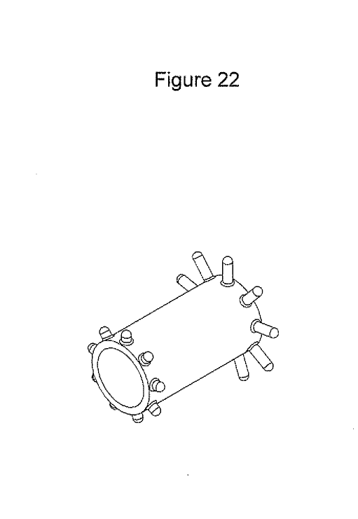

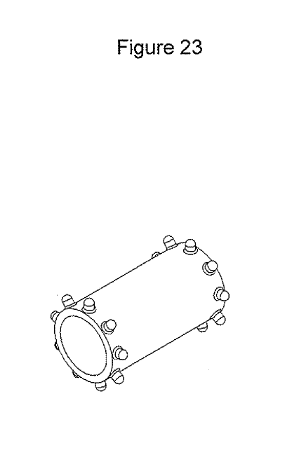

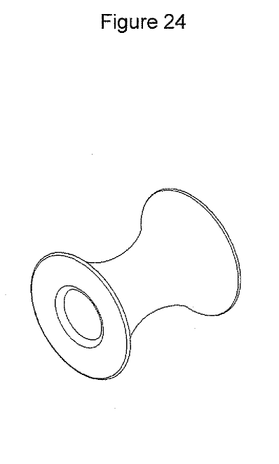

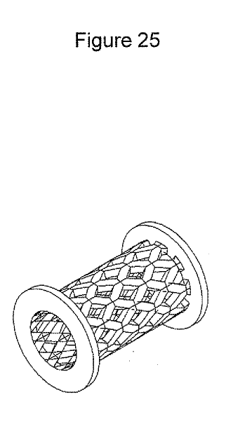

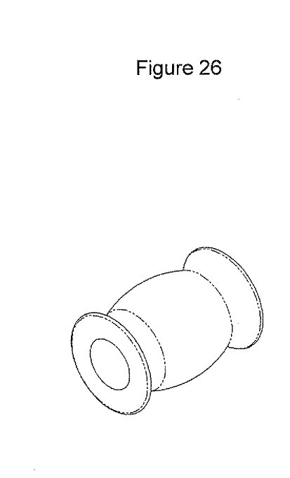

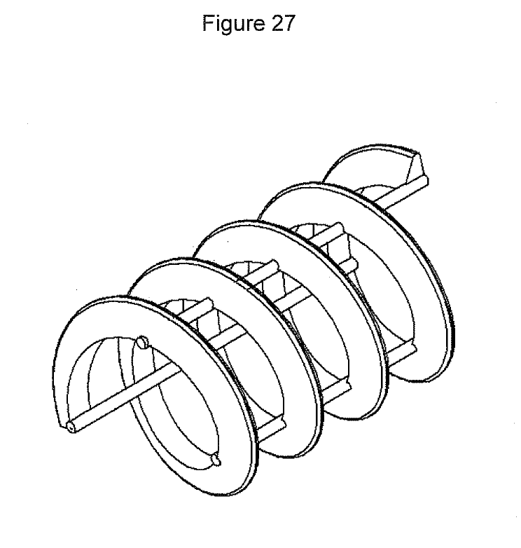

In certain embodiments, the tube includes flanges, ridges, or other protruding structures at one or both ends as shown in FIG. 1 to hold the tube in place and prevent it from falling into the sinus cavity or into the nasal passage. The flange, ridge, or other protruding structure typically protrudes approximately 0.5 mm or less. It may protrude approximately 0.1 mm, 0.2 mm, 0.3 mm, or 0.4 mm. The inner flange, ridge, or other structure may protrude less than the outer one which remains in the nasal passage. This difference between the two ends allows for easy insertion of the tube. Alternatively, the tube is symmetrical with the two ends being the same. The flange may be any shape. For example, it may be circular, rectangular, triangular, hexagonal, polygonal, linear, X-shaped, broken, etc. In certain embodiments, the flange is shaped differently on each end. Preferably, the inner flange is shaped so that it can be deformed or is otherwise easily inserted into the ostia of the sinus (e.g., the flange may be tapered). For example, two portions of the flange may be folded to allow for easy insertion of the tube. In certain embodiments, the inner flange is sloped or tapered for easy insertion into the ostia. In other embodiments, the flange or inner opening is radially compressed for insertion into the ostia. In certain embodiments, the tube includes a small ridge at the inner opening which is inserted into the sinus cavity and a flange at the outer opening. This construction prevents the tube from falling into the sinus cavity. Rather it is more likely to fall into the nasal passage when it is ready to come out. Various designs of tubes with flanges are shown in the FIGS. 7-9, 11-19, and 21-26.

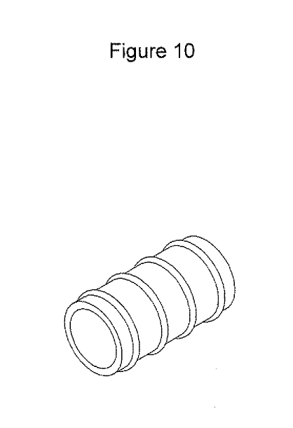

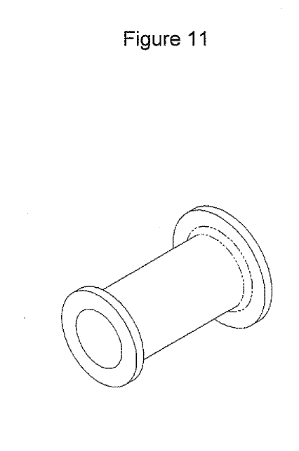

In certain embodiments, the outside surface of the sinus tube includes ridges, threads, crevices, or other irregularities to provide for holding the tube in place once it is inserted. See, e.g., FIG. 10. These irregularities on the surface may be microscopic or macroscopic. The irregularities on the surface typically provide holding the tube in place by increased friction. However, tissue or cells may grow into these irregularities providing an increased hold on the tube. The ridges or crevices may run in any direction, for example, parallel to the long axis of the tube, or perpendicular to the long axis of the tube. They may also run diagonally about the surface of the tube. They may also circle around the tube or spiral around the tube. In certain embodiments, the surface may be irregular with no clear pattern. The outside surface of the tube may also be coated with a material to provide an increased coefficient of friction (e.g., a polymeric coating).

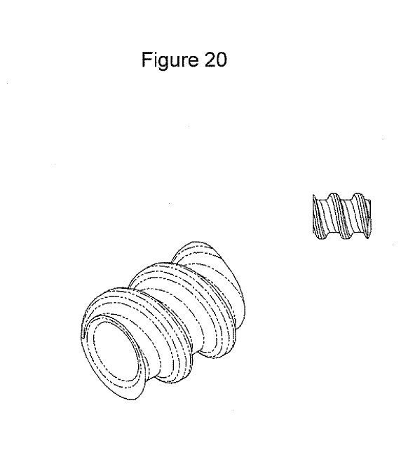

In certain particular embodiments, the outside surface of the tube includes a screw-like ridge so that the tube can be screwed into place in the ostia of the sinus, thereby preventing it from easily falling out. See, e.g., FIG. 20. The thread on the outside of the tube is typically only 0.1 to 1 mm in height. The thread provides enough traction to prevent the tube from falling out accidentally while the ostia is swollen. However, in certain embodiments, once the inflammation subsides, the tube may fall out of place and be harmlessly swallowed or sneezed out by the patient. The use of a screw-like mechanism to insert the tube allows for the tube to be designed without an inner and/or outer flange. Therefore, in certain embodiments, the screwed in sinus tube includes an outer flange but not an inner one. The lack of an inner flange allows for the unimpeded flow of mucus from the sinus cavity through the tube into the nasal passage. In certain embodiments, the screwed in sinus tube does not include an inner or outer flange. In other embodiments, the screwed in sinus tube includes both an inner and outer flange.

In certain embodiments, the sinus tube is coated. The coating may provide the release of a pharmaceutical agent, may prevent the adhesion of the tube in place, may prevent cell growth or scar formation, etc. The coating is preferably biocompatible. In certain embodiments, the coating is a polymeric coating. In certain embodiments, the coating is a polymeric coating that includes a therapeutic agent. Classes of therapeutic agents that may be delivered by the tube include DNA, RNA, nucleic acids, proteins, peptides, or small molecules. Exemplary therapeutic agents include antibiotics, anti-inflammatory agents, corticosteroids, vasoconstrictors, vasodilators, anti-allergy agents, anti-histamines, cromolyn sodium, decongestants, asthma treatments, etc. In certain embodiments, the coating include an antibiotic. The antibiotic prevents the microbial growth on the surface of the tube, as well as, optionally, releasing antibiotic into the surrounding area. In certain embodiments, the coating includes cytotoxic agents such as paclitaxel to prevent cell growth on the tube. In other embodiments, the coating is Teflon. The tube may be coated with a polysaccharide such as hyaluronate.

The tube can be made of any biocompatible material. The material may be synthetic (e.g., polyesters, polyanhydrides) or natural (e.g., proteins, rubber, polysaccharides). Preferably, the tube is made of a biodegradable material. In certain embodiments, the material is a biodegradable polymer. In certain embodiments, the material is a homopolymer. In certain embodiments, the material is a co-polymer. In other embodiments, the material is a block polymer. In other embodiments, the material is a branched polymer. In other embodiments, the material is a cross-linked polymer. In certain embodiments, the polymer is a polyester, polyurethane, polyvinyl chloride, polyalkylene (e.g., polyethylene), polyolefin, polyanhydride, polyamide, polycarbonate, polycarbamate, polyacrylate, polymethacrylate, polystyrene, polyurea, polyether, polyphosphazene, poly(ortho esters), polycarbonate, polyfumarate, polyarylate, polystyrene, or polyamine. In certain embodiments, the polymers is polylactide, polyglycolide, polycaprolactone, polydioxanone, polytrimethylene carbonate, and co-polymers thereof. Polymers that have been used in producing biodegradable implants and are useful in preparing the inventive tubes include alpha-polyhydroxy acids; polyglycolide (PGA); copolymers of polyglycolide such as glycolide/L-lactide copolymers (PGA/PLLA), glycolide/D,L-lactide copolymers (PGA/PDLLA), and glycolide/trimethylene carbonate copolymers (PGA/TMC); polylactides (PLA); stereocopolymers of PLA such as poly-L-lactide (PLLA), poly-D, L-lactide (PDLLA), L-lactide/D,L-lactide copolymers; copolymers of PLA such as lactide/tetramethylglycolide copolymers, lactide/trimethylene carbonate copolymers, lactide/δ-valerolactone copolymers, lactide ε-caprolactone copolymers, polydepsipeptides, PLA/polyethylene oxide copolymers, unsymmetrically 3,6-substituted poly-1,4-dioxane-2,5-diones; polyhydroxyalkanate polymers including poly-beta-hydroxybutyrate (PHBA), PHBA/beta-hydroxyvalerate copolymers (PHBA/HVA), and poly-beta-hydroxypropionate (PHPA); poly-p-dioxanone (PDS); poly-δ-valerolatone; poly-ε-caprolactone; methylmethacrylate-N-vinyl pyrrolidone copolymers; polyesteramides; polyesters of oxalic acid; polydihydropyrans; polyalkyl-2-cyanoacrylates; polyurethanes (PU); polyvinyl alcohol (PVA); polypeptides; poly-beta-maleic acid (PMLA); poly(trimethylene carbonate); poly(ethylene oxide)(PEO); poly(β-hydroxyvalerate) (PHVA); poly(ortho esters); tyrosine-derived polycarbonates; and poly-beta-alkanoic acids. In certain embodiments, the polymer is a polyester such as poly(glycolide-co-lactide) (PLGA), poly(lactide), poly(glycolide), poly(D,L-lactide-co-glycolide), poly(L-lactide-co-glycolide), poly-β-hydroxybutyrate, and polyacrylic acid ester. In certain embodiments, the tube is made of PLGA. In certain embodiments, the tube is made of 85% D,L-lactide and 15% glycolide co-polymer. In certain embodiments, the tube is made of 50% D,L-lactide and 50% glycolide co-polymer. In certain embodiments, the tube is made of 65% D,L-lactide and 45% glycolide co-polymer. In certain embodiments, the tube is made of 75% D,L-lactide and 25% glycolide co-polymer. In certain embodiments, the tube is made of 85% L-lactide and 15% glycolide co-polymer. In certain embodiments, the tube is made of 50% L-lactide and 50% glycolide co-polymer. In certain embodiments, the tube is made of 65% L-lactide and 45% glycolide co-polymer. In certain embodiments, the tube is made of 75% L-lactide and 25% glycolide co-polymer. In certain emboidments, the tube is made of poly(caprolactone). In certain embodiments, the tube is made of Pebax, Polyimide, Braided Polyimide, Nylon, PVC, Hytrel, HDPE, or PEEK. In certain embodiments, the tube is made of a fluoropolymer such as PTFE, PFA, FEP, and EPTFE. In certain embodiments, the tube is made of latex. In other embodiments, the tube is made of silicone. The polymer typically has a molecular weight sufficient to be shaped by molding or extrusion. The tube may be made of a material that is bioabsorbed after the tube is not longer needed. The polymer used may be selected based on its degradation profile. For example, the tube may degrade after 1 week, 2 weeks, 3 weeks, 1 month, 2 months, 3 months, 4 months, 5 months, 6 months, 12 months, 18 months, 24 months, etc. In certain embodiments, the degradation period is up to about 2 years; or between about 3 weeks and about 1 year; or between about 6 weeks and about 9 months. The tube may also be made of a material that is degrade harmlessly in the digestive tract of the patient. Therefore, when the tube falls out and is swallowed, the tube is harmlessly degraded and/or eliminated by the patient.

In other embodiments, the tube is made of a metal. In other embodiments, the tube is made of an alloy. In certain embodiments, the tube is made of stainless steel. In certain embodiments, the tube is made of a magnesium alloy (e.g., magnesium based alloy AE21). See, e.g., Heublein et al., “Biocorrosion of magnesium alloys: a new principle in cardiovascular implant technology?” Heart 89:651-56, 2003; incorporated herein by reference. Metal tubes may be optionally coated with a biocompatible coating. In the case where the tube is made of a metal, the tube may be inserted permanently or may be removed manually after the tube is no longer needed.

Other medical tubes are known in the medical arts. Such tubes include esophageal tubes, gastroinstestinal tubes, vascular tubes, biliary tubes, Eustachian tube tubes, bronchial tubes, tracheal tubes, etc. The use and construction of these other types of tubes is applicable to the use and construction of the inventive sinus tubes. Representative examples of tubes include those discussed in U.S. Pat. Nos. 4,768,523; 4,776,337; 5,041,126; 5,052,998; 5,064,435; 5,089,606; 5,247,370; 5,176,626; 5,213,580; 5,246,455; 5,693,065; each of which is incorporated herein by reference.

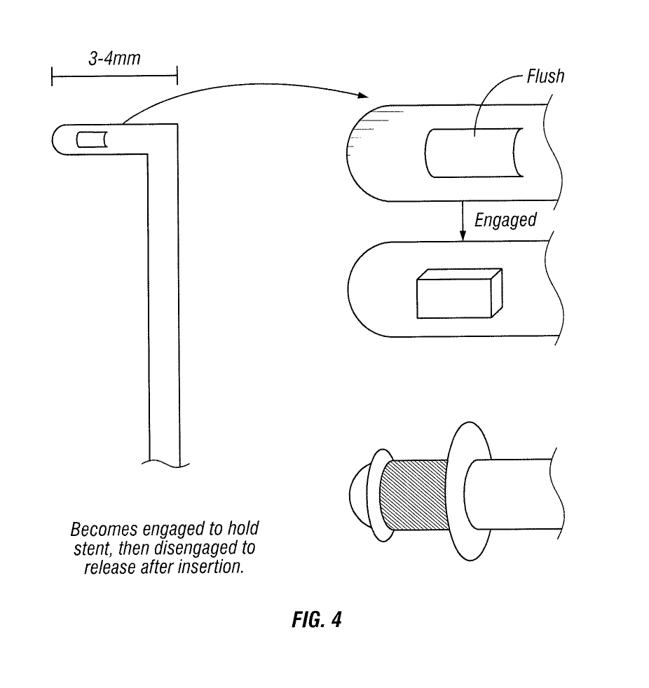

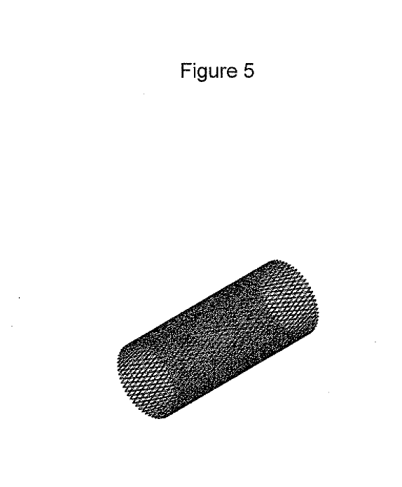

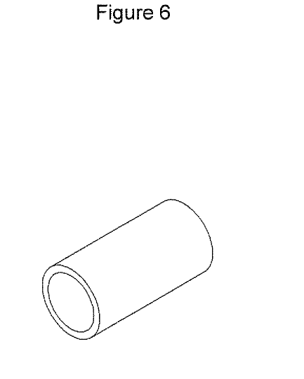

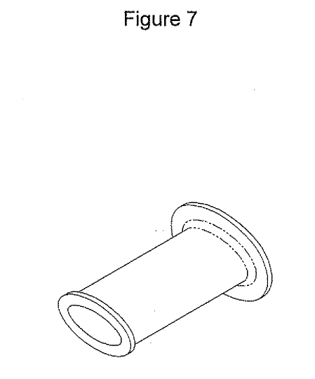

The invention also provides an instrument for inserting the inventive sinus tubes into the ostia of a sinus. The instrument may include a comfortable handle with a triggering mechanism for engaging and disengaging the inventive tubes and an elongated tip for inserting into the nose of the patient and into the ostia. A tube is typically placed on the end of the instrument, and the instrument is engaged to firmly hold the tube while it is being maneuvered through the nasal passage and into the ostia of the sinus. Once the instrument with the tube is in place in the ostia of the sinus, the tube is released and the instrument is then withdrawn. FIG. 4 shows an exemplary instrument with a piece that protrudes from the tip to hold/engage the tube. Once the instrument with the engaged tube is in place, the protruding piece is triggered to become flush with the surface of the instrument and the tube is released. As would be appreciated by one of the skill in the art, other means for engaging and disengaging the tube from the instrument could also be used in designing the instrument. For example, a protruding ring could be used to engage the tube on the tip of the instrument.

The inventive tube may be packaged in kits for convenience. In certain embodiments, the kits may also include all or some of the following items: an instrument for inserting the tube into the ostia, an instrument for removing the tube, pharmaceutical agents, nasal sprays, gauze, disinfectant, and instructions for using the tube and the instrument. In certain embodiments, the kits are sterilely package for convenient use by a surgeon or other physician.

What is claimed is:

1. An implantable tube for insertion into the wall of a nasal cavity for venting a sinus comprising:
a tubular structure having first and second open ends and a lumen of a diameter of 0.5 mm to 5 mm extending there between and of a length of 0.5 mm to 3 mm;
a first flange integrally formed on first said opening; and
a second flange integrally formed on second said opening.

2. The tube of claim 1, wherein the tube is one piece.

3. The tube of claim 1, wherein the tube is molded.

4. The tube of claim 1, wherein the tube is extruded.

5. The tube of claim 1, wherein the tube is flexible.

6. The tube of claim 1, wherein the tube is rigid.

7. The tube of claim 1, wherein the tube is made of a biodegradable material.

8. The tube of claim 7, wherein the biodegradable material is absorbed in approximately 3-4 months.

9. The tube of claim 7, wherein the biodegradable material is absorbed in approximately 6 months.

10. The tube of claim 7, wherein the biodegradable material is absorbed in approximately 12 months.

11. The tube of claim 1, wherein the tube is made of a non-biodegradable material.

12. The tube of claim 1, wherein the tube is made of silicone.

13. The tube of claim 1, wherein the tube is made of a metal or alloy.

14. The tube of claim 1, wherein the tube is made of a polymer.

15. The tube of claim 1, wherein the tube is made of a biodegradable, biocompatible polymer.

16. The tube of claim 1, wherein the tube is made of a polymer selected from the group consisting of polyesters, polyurethanes, polyvinyl chloride, polyalkylenes, polyolefins, polyanhydrides, polyamides, polycarbonates, polycarbamates, polyacrylates, polymethacrylates, polystyrenes, polyureas, polyethers, polyphosphazenes, poly(ortho esters), polycarbonates, polyfumarates, polyarylates, polystyrenes, or polyamines.

17. The sent of claim 1, wherein the tube is made of PLGA.

18. The tube of claim 1, wherein the tube is made of a polyester.

19. The tube of claim 1, wherein the tube is silastic.

20. The tube of claim 1, wherein the sinus being vented is the maxillary sinus.

21. The tube of claim 1, wherein the sinus being vented is the sphenoid sinus.

22. The tube of claim 1, wherein the sinus being vented is the frontal sinus.

23. The tube of claim 1, wherein the tube is capable of being inserted without destruction of tissue around the natural ostia of the sinus.

24. The tube of claim 1, wherein the diameter of the first flange is 1.2 times the diameter of the lumen.

25. The tube of claim 1, wherein the diameter of the second flange is 1.2 times the diameter of the lumen.

26. The tube of claim 1, wherein the diameter of the second flange is 1.5 times the diameter of the lumen.

27. The tube of claim 1, wherein the diameter of the second flange is 2 times the diameter of the lumen.

28. The tube of claim 1, wherein the first flange is circular shaped, linear shaped, X-shaped, triangular shaped, rectangular shaped, pentagonal shaped, or hexagonal shaped.

29. The tube of claim 1, wherein the second flange is circular shaped, linear shaped, X-shaped, triangular shaped, rectangular shaped, pentagonal shaped, or hexagonal shaped.

30. The tube of claim 1, wherein the tube is coated.

31. The tube of claim 30, wherein the tube is coated with Teflon.

32. The tube of claim 30, wherein the tube is coated with hyaluronidate.

33. The tube of claim 30, wherein the coating is collagen.

34. The tube of claim 30, wherein the coating is a polymer.

35. The tube of claim 30, wherein the coating comprises a pharmaceutical agent.

36. The tube of claim 30, wherein the tube comprises a pharmaceutical agent.

37. The tube of claim 35 or 36, wherein the pharmaceutical agent is selected from the group consisting of decongestants, anti-histamines, corticosteroids, anti-inflammatory agents, and antibiotics.

38. An threaded, implantable tube for insertion into the wall of a nasal cavity for venting a sinus comprising a tubular structure having first and second open ends and a lumen of a diameter of 0.5 mm to 5 mm extending there between and of a length of 0.5 mm to 3 mm, wherein the tubular structure include a screw-like thread running along the long axis of the structure on the outside of the structure.

39. A device for introducing the tube of claim 1 into a an ostia of a sinus comprising:
a handle; and
an elongated tip for engaging the tube to be inserted and then disengaging the tube once the tube has been place in an ostia.

40. A system comprising a tube of claim 1 and a device of claim 39.

41. A method of introducing a tube of claim 1 into a subject, the method comprising steps of:
visualizing the ostia into which the tube is to be inserted;
introducing the tube into the ostia using the device of claim 39; and
disengaging the tube from the device.

42. The method of claim 41, wherein the step of introducing comprising introducing the tube to a natural ostia, wherein no tissue has been removed from around the ostia.

43. The method of claim 41 further comprising the step of performing a uncinectomy.

44. A kit comprising a sinus tube of claim 1.

45. The kit of claim 44 further comprising an instrument for inserting the tube in an ostia of a paranasal sinus.

46. The kit of claim 44 further comprising instructions for using the sinus tube.
